(19) United States
(12) Patent Application Publication
(10) Pub. No.: US 2024/0002771 A1
(43) Pub. Date: Jan. 4, 2024

(54) CELL CULTURE MEDIA CONDITIONING VESSELS AND PERFUSION BIOREACTOR SYSTEM

(71) Applicant: CORNING INCORPORATED, Corning, NY (US)

(72) Inventors: Stephen Joseph Caracci, Elmira, NY (US); Joel Andrew Schultes, CORNING, NY (US)

(21) Appl. No.: 18/039,365

(22) PCT Filed: Nov. 19, 2021
(86) PCT No.: PCT/US2021/060040

§ 371 Date: May 30, 2023

Related U.S. Application Data
(60) Provisional application No. 63/119,007, filed on Nov. 30, 2020.
(60) Provisional application No. 63/119,045, filed on Nov. 30, 2020.

(51) Int. Cl. C12M 1/00; C12M 1/34

(52) U.S. Cl. CPC C12M 29/26; C12M 29/10; C12M 41/12; C12M 41/26; C12M 41/34; C12M 29/06

(57) ABSTRACT

A media conditioning vessel is provided for a perfusion bioreactor system. The media conditioning vessel includes a vessel having an interior cavity to hold a volume of liquid cell culture media; a media inlet to return cell culture media from a perfusion bioreactor to the vessel; and a media outlet to transfer cell culture media out of the vessel to the perfusion bioreactor. Sensors to measure or detect a characteristic of the cell culture media are provided in the form of inline sensors in at least one of the media inlet and the media outlet or patch sensors attached to a sidewall or bottom of the media conditioning vessel.

### CROSS REFERENCE TO RELATED APPLICATIONS

This application claims the benefit of priority under 35 U.S.C. § 119 of U.S. Provisional Application Ser. No. 63/119,007 filed on Nov. 30, 2020 and U.S. Provisional Application Ser. No. 63/119,045 filed on Nov. 30, 2020 the content of which is relied upon and incorporated herein by reference in its entirety.

### FIELD OF THE DISCLOSURE

This disclosure generally relates to media conditioning systems for cell culture system, and bioreactors and bioreactor systems for cell culture with media conditioning systems.

### BACKGROUND

In the bioprocessing industry, large-scale cultivation of cells is performed for purposes of the production of hormones, enzymes, antibodies, vaccines, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells. Alternative methods have been suggested to increase volumetric density of cultured cells. These include microcarrier cultures performed in stir tanks. Another example of a high-density cell culture system is a hollow fiber bioreactor, in which cells may form large three-dimensional aggregates as they proliferate in the interspatial fiber space.

Yet another example of a high-density culture system for anchorage dependent cells is a packed-bed bioreactor system. In this this type of bioreactor, a cell substrate is used to provide a surface for the attachment of adherent cells. Medium is perfused along the surface or through the semi-porous substrate to provide nutrients and oxygen needed for the cell growth. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer. Such bioreactors function as recirculation flow-through bioreactors.

In these various types of cell culture reactors, the cells must be grown under controlled conditions, including being suspended or perfused in a cell culture media, which is a liquid media containing nutrients necessary for cell life and growth. The contents of the cell culture media, including the pH, dissolved gas ratios, nutrients, and waste, as well as the temperature of the media and/or the cells, must be monitored and controlled to optimize cell growth and other performance of the bioreactor. Therefore, media conditioning systems are used in combination with the bioreactors to condition the media therein. Traditionally, media conditioning has been integrated into the cell culture vessel itself. In other words, media conditioning and/or mixing of the cell culture media occurs in the same vessel where the cells are cultured. Media conditioning is often performed in a hollow straight-walled container or vessel (e.g., a beaker or bottle) with a complicated system of probes to control the composition of the media, one or more stirrers to mix the media, and some type of temperature control jacket around the vessel. The probes may enter the vessel through a cap on the vessel body, and sterility is always a concern, especially if the system is open or opened during use.

However, there is a need for improved media conditioning systems that can meet the demands of scalable cell culturing platforms, while providing a simple, reliable, and closed architecture.

### SUMMARY

According to an embodiment of this disclosure, a media conditioning vessel for a perfusion bioreactor system is provided that includes a vessel comprising an interior cavity configured to hold a volume of liquid cell culture media; a media inlet configured to return cell culture media from a perfusion bioreactor to the vessel; and a media outlet configured to transfer cell culture media out of the vessel to the perfusion bioreactor, wherein at least one of the media inlet and the media outlet comprises one or more inline sensors configured to measure or detect a characteristic of the cell culture media.

In aspects of some embodiments, the one or more inline sensors comprises at least one of a dissolved oxygen sensor, a pH sensor, and a temperature sensor. The media conditioning vessel further includes a gas sparging tube configured to sparge a gas in the interior cavity. The media conditioning vessel can further include a perfusion loop comprising a pump configured to pump cell culture media from the media outlet to a media return that returns cell culture media to the interior cavity.

In some embodiments, a media conditioning vessel for a perfusion bioreactor system is provided that includes: a vessel comprising an interior cavity configured to hold a volume of liquid cell culture media; a media inlet configured to return cell culture media from a perfusion bioreactor to the vessel; a media outlet configured to transfer cell culture media out of the vessel to the perfusion bioreactor; and one or more patch sensors attached to at least one of a sidewall or a bottom of the media conditioning vessel and configured to measure or detect a characteristic of the cell culture media. The one or more patch sensors can include at least one of a dissolved oxygen sensor, a pH sensor, and a temperature sensor.

Additional aspects of the present disclosure will be set forth, in part, in the detailed description, figures and any claims which follow, and in part will be derived from the detailed description, or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

FIG. 1 shows a schematic of a cell culture system according to one or more embodiments of this disclosure.

FIG. 2 shows a representation of a media conditioning vessel with inline sensors, according to one or more embodiment.

FIG. 3 shows a representation of a media conditioning vessel with inline sensors and a recirculation loop, according to one or more embodiment.

FIG. 4 shows a representation of a media conditioning vessel with patch sensors, according to one or more embodiment.

FIG. 5 shows a close-up representation of a gas sparge line and media inlet of a media conditioning vessel, according to one or more embodiment.

FIG. 6 shows a representation of a media conditioning vessel, according to one or more embodiment.

FIG. 7 shows a representation of a media conditioning vessel, according to one or more embodiment.

FIG. 8A is a drawing of the geometry of a standard media conditioning vessel.

FIG. 8B is a drawing of the geometry of a standard media conditioning vessel.

FIG. 8C is a drawing of a media conditioning vessel geometry, according to one or more embodiment.

FIG. 8D is a drawing of a media conditioning vessel geometry, according to one or more embodiment.

FIG. 8E is a drawing of a media conditioning vessel geometry, according to one or more embodiment.

FIG. 9A is a drawing of a media conditioning vessel geometry, according to one or more embodiment.

FIG. 9B is a drawing of a media conditioning vessel geometry, according to one or more embodiment.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

Embodiments of this disclosure are directed to media conditioning vessels and systems, and cell culture systems incorporated media conditioning systems. In particular, the media conditioning systems described herein include media conditioning vessels that are separate from the vessels used for cell culture. The media conditioning vessels and systems can include a variety of sensors, such as inline and patch sensors, and improved or simplified designs for improved oxygenation of cell culture media and decreased risk of contamination. In some embodiments, it is contemplated that the bioreactor is a fixed bed or packed bed bioreactor with a high density scaffold for cell growth.

Many bioreactor vessels used, for example, for suspension cell culture are also used as the media conditioning vessel. As such, the vessel is designed to reduce sheer forced experienced by the cells and will contain specialized fixed sensors or probes in the media to monitor cell culture parameters. However, many of these features are not required in embodiments of this disclosure due to the media conditioning vessel being separated from the cell culture bioreactor vessel. This design, free from the constraints of conditioning media within the cell culture vessel, also allows for increased performance and a reduction in complexity and/or costs. For example, cell culture bioreactors with integrated media conditioning have to be highly engineered to maintain the bioreactor as a closed system to prevent contamination, while simultaneously allowing multiple probes/sensors to penetrate the vessel and/or media to monitor cell culture progress. The required level of engineering can be cost prohibitive for a single use vessel.

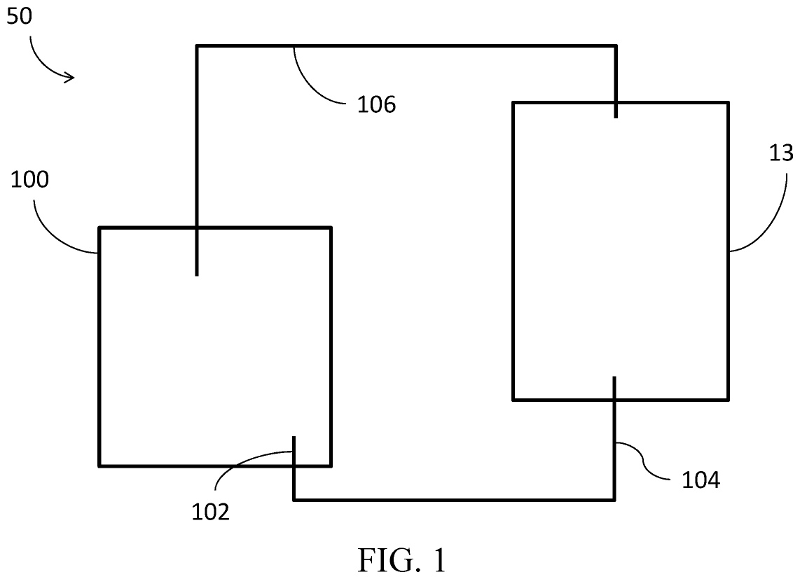

FIG. 1 shows a schematic of a cell culture system 50, according to one or more embodiments of this disclosure. The cell culture system 50 includes both a bioreactor vessel 13 for growing cells, cell by-products and/or viral vectors, and a media conditioning system 100 to condition media that is supplied to the bioreactor vessel. As shown in FIG. 1, the system 50 can be configured in a recirculation loop, where media flows out of the media conditioning system 100 via on outlet 102 to the bioreactor vessel 13 via tubing 104 or other fluid connector. Within the bioreactor vessel 13, the media supplies necessary nutrients to the cells and maintains a healthy cell environment. Optionally, the media can be returned to the media conditioning vessel 100 via a return tubing 106 or another connector. Embodiments of this disclosure include the entire cell culture system 50, as well as the individual media conditioning system 100, which can be used to condition media in a variety of systems or applications.

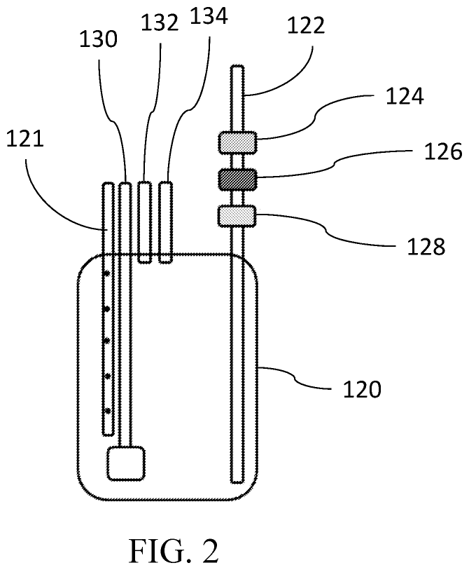

FIG. 2 is a schematic of a media conditioning vessel 120 for a perfusion bioreactor system, according to one or more embodiments of this disclosure. The media conditioning vessel 120 includes a media inlet 121 and a media outlet 122 for transferring cell culture media to and from the media conditioning vessel 120, respectively. Three in-line sensors are provided on the media outlet 122: a dissolved oxygen (DO) sensor 124, a pH sensor 126, and a temperature sensor 128. The media conditioning vessel 120 further includes a gas sparge 130, vent 132, and supplement inlet 134. The system in FIG. 2 reduces the number of penetrations in the vessel cap by using in-line sensors for monitoring the media at the exit of the vessel as it travels to the perfusion bioreactor.

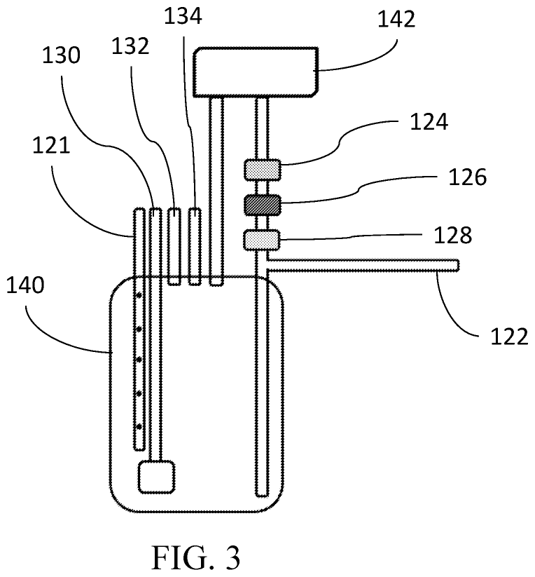

FIG. 3 shows a media conditioning vessel 140 according to one or more embodiments. Like the media conditioning vessel 120 of FIG. 2, the media conditioning vessel 140 includes a media inlet 121 and a media outlet 122 for transferring cell culture media to and from the media conditioning vessel 120, respectively. Three in-line sensors are provided on a perfusion loop connected to the media outlet 122: a dissolved oxygen (DO) sensor 124, a pH sensor 126, and a temperature sensor 128. The media conditioning vessel 140 further includes a gas sparge 130, vent 132, and supplement inlet 134. However, unlike the system in FIG. 2, the media conditioning vessel 140 also has a built-in perfusion loop to continuously sample media, using the in-line sensors, even when the downstream perfusion flow (via media outlet 122) has stopped.

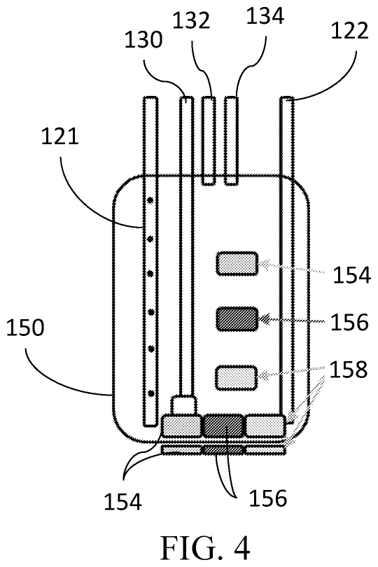

In FIG. 4, a media conditioning vessel 150 is provided according to another embodiment of this disclosure. Like the media conditioning vessel 120 of FIG. 2, the media conditioning vessel 150 includes a media inlet 121 and a media outlet 122 for transferring cell culture media to and from the media conditioning vessel 150, respectively. The media conditioning vessel 150 further includes a gas sparge 130, vent 132, and supplement inlet 134. Rather than the in-line sensors of FIGS. 2 and 3, the media conditioning vessel 150 uses patch sensors 154, 156, 158 provided on the media conditioning vessel 150: a dissolved oxygen (DO) sensor 154, a pH sensor 156, and a temperature sensor 158. Three different locations for each type of patch sensor are shown in FIG. 4: a vertical arrangement of sensors on a sidewall of the media conditioning vessel 150; a horizontal arrangement of sensors on the sidewall; and on the bottom of the vessel 150. As an aspect of some embodiments, patch sensors placed on the bottom of the media conditioning vessel 150 can make the assembly of the system (i.e., the placement of the sensors 154, 156, 158) easier and the media conditioning vessel 150 could be correctly oriented on a docking station to hold the readouts (e.g., optical readouts) of the patch sensors. This would also enable the use of a bottom magnetic stirrer, provided the patch sensors on the bottom on the media conditioning vessel 150 are not in the way of the magnetic sensor (e.g., the sensors 154, 156, 158) are located on a peripheral portion of the bottom of the media conditioning vessel 150. While FIGS. 2 and 3 show only inline sensors and FIG. 4 shows only patch sensors, embodiments of this disclosure include media conditioning vessels having a combination of in-line and patch sensors.

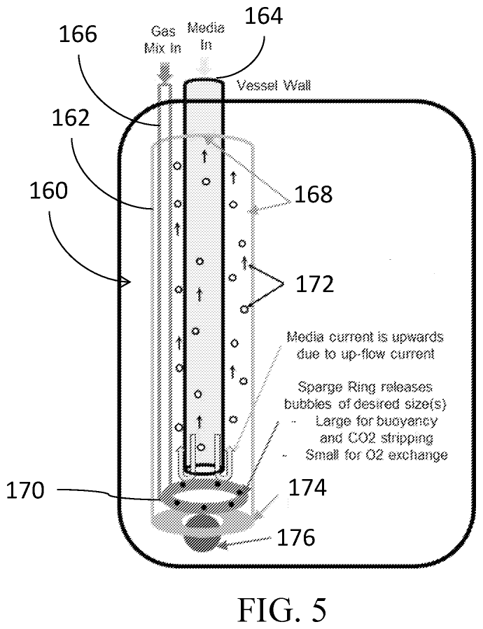

FIG. 5 is a magnified view (not to scale) of a gas sparge system 160 having an outer gas sparge tube 162 around a media inlet 164 and a gas inlet 166. The outer gas sparge tube 162 can include small holes or slits 168 that are too small for bubbles to pass through but large enough for cell culture media to drain from the outer gas sparge tube 162 and into the larger cell culture media reservoir of the media conditioning vessel. The holes or slits 168 may also help pop bubbles within the outer gas sparge tube 162. Gas flows through the gas inlet 166 to a sparge ring 170 near the bottom of the outer gas sparge tube 162. Gas bubbles 172 then escape from the sparge ring 170 and float upwards within the outer gas sparge tube 162, creating an up-flow current within the outer gas sparge tube 162. Media existing the bottom of the media inlet 164 then also flows upward due to the up-flow current. The sparge ring 170 can also release bubbles of a desired size depending on a desired function to be performed. For example, large bubbles can be released for buoyancy and CO₂ stripping, while small bubbles can be released for O₂ exchange. The outer gas sparge tube 162 has an opening 174 for drainage. The opening 174 can be equipped with a drain plug 176 that closes at a high media level and drops at a lower media level. By introducing spent media into the confined space within the outer gas sparge tube 162, more efficient sparging can occur and sparge bubbles can be kept way from the outlet of the media conditioning vessel, thereby preventing the bubbles from entering the bioreactor vessel.

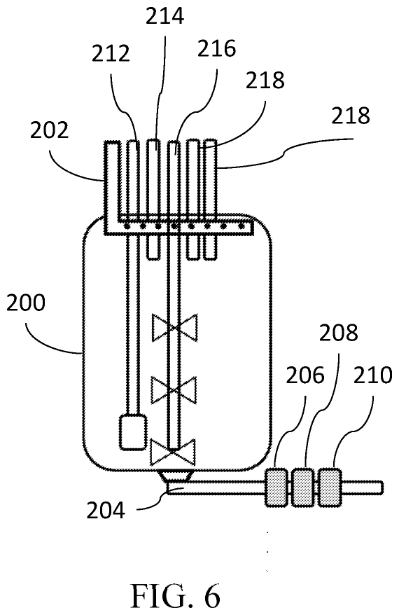

FIG. 6 shows a media conditioning vessel 200 with a top media inlet 202 and a bottom media outlet 204. Advantages of the top media inlet 202 is that media can be dispersed over the inner walls of the vessel 200 (e.g., dripped or sprayed) utilizing the gas mixture in the overlay portion of the vessel 200 to help oxygenate the media. As the media flows into the vessel, a high surface area to volume (media film or rivulets) can be achieved for pre-oxygenation of the media prior to reaching the bulk volume. Agitation can be used for mixing, or an open-bottom gas sparging tube could be used for mixing. The bottom media outlet 204 has the advantage of allowing lower hang-up of liquid in the vessel 200, and provides a higher liquid pressure in the media outlet 204 to prevent out-gassing and subsequent bubble formation upstream of the pump.

The media conditioning vessel 200 also includes a DO inline sensor 206, a pH inline sensor 208, and a temperature inline sensor 210. Further, the media conditioning vessel 200 can have a gas sparging line 212, a vent 214, a mixer 216, a supplement in 218, and a gas overlay tube 220.

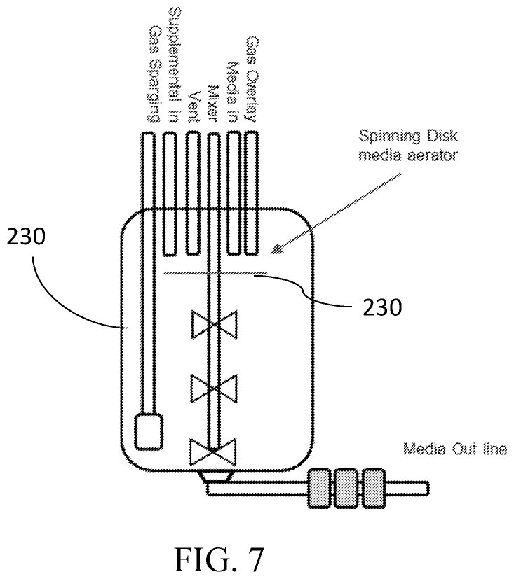

FIG. 7 shows a media conditioning vessel 230 that is a variation on the media conditioning vessel 200 of FIG. 6. The media conditioning vessel 230 includes a gas sparging line 231, supplemental inlet 238, media inlet 233, vent 234, mixer 236, gas overlay line 239, and a spinning disk aerator 232. The spent media impinges upon the disk 232, which is spinning, and creates small droplets of media to be oxygenated in the overlay portion of the media conditioning vessel 230. This spinning disk 230 can be on the media mixer 236, or on its own rotating shaft.

As discussed above, media conditioning for perfusion bioreactor systems have fewer demands on them with regard to cell shear, sensing, and mixing. Because of this, vessel designs beyond the traditional straight-sidewall vessels are more possible and can even provide additional benefits. Accordingly, embodiments of this disclosure include vessels with new and unique sidewall geometries that enable different minimum volumes of media to be used in perfusion systems. Furthermore, the same media conditioning vessels can be used to allow for a larger range of perfusion systems, can enable a larger air/media interface to increase gas exchange at the surface without relying on sparging, which can cause undesirable foaming in the cell culture media. Embodiments of this disclosure include media conditioning vessels with larger turndown ratios than traditional straight-sidewall vessels. “Turndown ratio” refers to the range of capacity serviceable by a given component (in this case, by a media conditioning vessel). In other words, the turndown ratio is a ratio of a maximum capacity to a minimum capacity. Larger turndown ratios allow a user to use the same media conditioning vessel for differently sized perfusion systems.

Embodiments of this disclosure include media conditioning vessels of various different geometries that can allow them to be used in different implementations and some can be used over a wider range of perfusion bed sizes because of its range of volumes (i.e., the turndown ratio of the media conditioning vessel), and can be used to increase the surface area of the overlay interface (i.e., gas/media interface) for improved gas exchange.

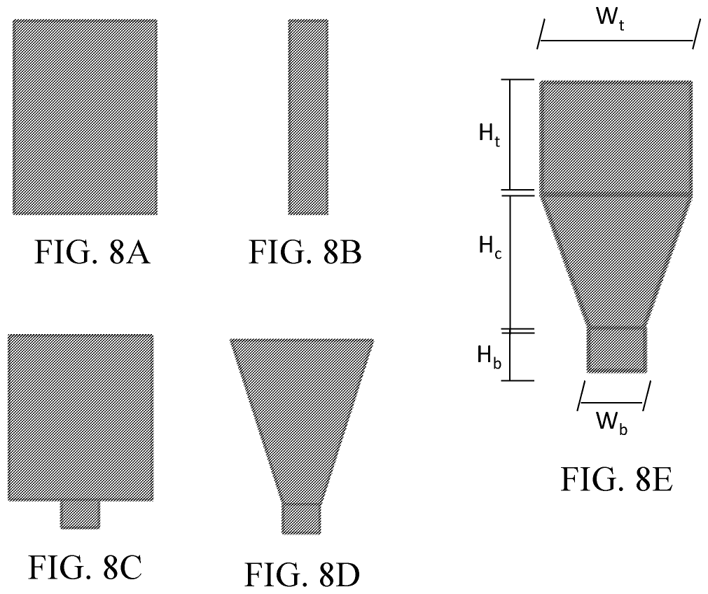

FIGS. 8A-8E show various geometries for media conditioning vessels, according to some embodiments. FIG. 8A shows an example of a traditional bioreactor vessel for the media conditioning vessel. The volume of the media (or container) increases linearly with increasing media height in the vessel. This has the distinct disadvantages of having a large minimum working volume, and the surface area of the gas/media interface does not increase with addition of media volume. Although this is partially offset by the fact that the surface area to volume ratio might be large enough at small volumes to enable what is called overlay gassing to dissolve gasses into the media.

FIG. 8B shows how the media conditioning vessel geometry can be changed to reduce the minimum working volume, but then suffers from a lower total working volume and the surface area again does not scale with increasing amount of media. In this case the surface area to volume ratio is very small, and overlay gassing will not work at much more than the minimum working volume.

FIG. 8C is an example of an embodiment where the minimum working can be reduced while keeping a large total working volume for the vessel. Like the example of FIG. 8A, the surface area to volume ratio would allow efficient overlay gassing to a certain volume of media.

FIG. 8D is another example of an embodiment that uses a cone-shaped conditioning vessel. A low minimum working volume is achieved due to the small dimensions at the bottom portion, and then the surface area to volume ratio increases as additional media is added. This has the two-fold benefit of having a larger total working volume, and allowing an increasing surface area to volume ratio to keep overlay gassing an option up to the maximum working volume.

FIG. 8E shows another geometry that may be more practical in some case while still incorporating the cone shape of FIG. 8D. The vessel of FIG. 8E has a top portion defined by a top width Wₜ and a top height Hₜ, a cone portion with a cone height H_{c}, and a bottom portion with a bottom width W_{b} and a bottom height H_{b}. The top portion with vertical sidewalls above the cone portion allows for the vessel to have a headspace of overlay gasses and a geometry more conducive to having a head plate with probes and stirring agitators mounted. Table 1 shows eight example media conditioning vessels defined in terms of top width Wₜ, top height Hₜ, cone height H_{c}, bottom with W_{b}, bottom height H_{b}, and the minimum and total working volumes.

TABLE 1
Minimum and Maximum working volumes for various
media conditioning vessel geometries.

| | | | | | | Minimum | Maximum |
|---|---|---|---|---|---|---|---|
| | T_{w} | T_{b} | H_{b} | Hc | Hₜ | Working | Working |
| Sample | (cm) | (cm) | (cm) | (cm) | (cm) | Volume (L) | Volume (L) |
| A | 5 | 5 | 0 | 0 | 25 | 0.31 | 2.0 |
| B | 8 | 5 | 4 | 21 | 0 | 0.31 | 3.1 |
| C | 20 | 5 | 5 | 0 | 20 | 0.31 | 25.5 |
| D | 20 | 20 | 0 | 0 | 25 | 5.03 | 31.4 |
| E | 5 | 5 | 5 | 0 | 320 | 0.31 | 25.5 |
| F | 20 | 5 | 5 | 5 | 17.8 | 0.31 | 25.5 |
| G | 20 | 5 | 5 | 0 | 20 | 0.31 | 25.5 |
| H | 20 | 20 | 0 | 0 | 20.3 | 5.03 | 25.5 |

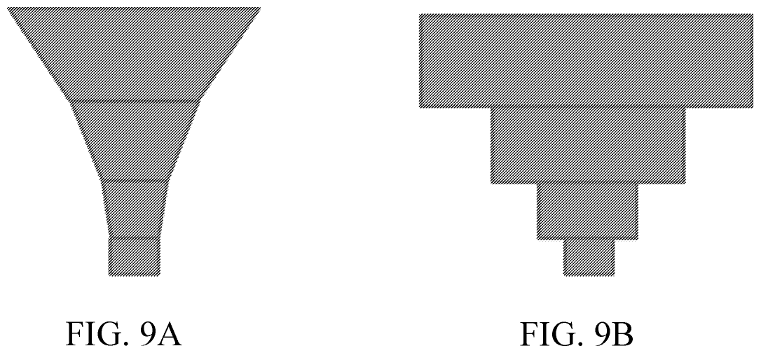

FIGS. 9A and 9B show two media conditioning vessel configurations that can be used with a perfusion fixed bed, according to some embodiments. In an aspect of some embodiments, the perfusion fixed bed can be scaled to different capacities by increasing a height of the packed bed and/or a number of layers of substrate in the packed bed stack. The different vertical sections in FIGS. 9A and 9B would be used with an increasing number of cell culture layers in the packed bed. The larger top radius (or size) provides an increase in surface area to maintain a constant surface area to volume ratio, so that the same gassing schema can be used, and the volume also increases to allow for the additional media volume required for the additional cell number that would come with increased bed size. It should be noted that the cross section of the vessels can be circular, rectangular or square to accommodate surface area to volume ratios from the bottom of the vessel to the top.

In some embodiments, the media conditioning system further includes one or more sensors for sensing a property of the gas within the enclosure, or of the media within the gas exchange system, or of the media prior to entering, after exiting, or while within the bioreactor. The one or more sensors can measure temperature, pH, oxygen (O₂), CO₂, or any of a number of variables that are relevant to the cell culture operation being performed.

It is contemplated that the media conditioning vessels and systems disclosed herein may be used with a bioreactor having a packed-bed of fixed-bed cell culture substrate. In conventional large-scale cell culture bioreactors, different types of packed bed bioreactors have been used. Usually these packed beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped or entangled in fibers of the matrix. Thus, because of linear flow of the cell inoculum through the packed bed, cells are subject to heterogeneous distribution inside the packed-bed, leading to variations in cell density through the depth or width of the packed bed. For example, cell density may be higher at the inlet region of a bioreactor and significantly lower nearer to the outlet of the bioreactor. This non-uniform distribution of the cells inside of the packed-bed significantly hinders scalability and predictability of such bioreactors in bioprocess manufacturing, and can even lead to reduced efficiency in terms of growth of cells or viral vector production per unit surface area or volume of the packed bed.

Another problem encountered in packed bed bioreactors disclosed in prior art is the channeling effect. Due to random nature of packed nonwoven fibers, the local fiber density at any given cross section of the packed bed is not uniform. Media flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products. Thus, the non-uniformity leads to areas with different exposure to flow and shear, effectively reducing the usable cell culture area, causing non-uniform culture, and interfering with transfection efficiency and cell release.

To address these and other problems of existing cell culture solutions, embodiments of the present disclosure provide cell growth substrates, matrices of such substrates, and/or packed-bed systems using such substrates that enable efficient and high-yield cell culturing for anchorage-dependent cells and production of cell products (e.g., proteins, antibodies, viral particles). Embodiments include a porous cell-culture matrix made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

In one embodiment, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed matrix, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of one or more embodiments enables complete cell recovery and consistent cell harvesting from the packed bed of the bioreactor.

According to some embodiments, a method of cell culturing is also provided using bioreactors with the matrix for bioprocessing production of therapeutic proteins, antibodies, viral vaccines, or viral vectors.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting. According to one or more particular embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about g 10¹⁶ viral genomes per batch. In some embodiments, production is about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

The cell culture substrate can be a woven mesh layer made of a first plurality of fibers running in a first direction and a second plurality of fibers running in a second direction. The woven fibers of the substrate form a plurality of openings, which can be defined by one or more widths or diameters. The size and shape of the openings can vary based on the type of weave (e.g., number, shape and size of filaments; angle between intersecting filaments, etc.). A woven mesh may be characterized as, on a macro-scale, a two-dimensional sheet or layer. However, a close inspection of a woven mesh reveals a three-dimensional structure due to the rising and falling of intersecting fibers of the mesh. Without wishing to be bound by theory, it is believed that the three-dimensional structure of the substrate is advantageous as it provides a large surface area for culturing adherent cells, and the structural rigidity of the mesh can provide a consistent and predictable cell culture matrix structure that enables uniform fluid flow.

In one or more embodiments, a fiber may have a diameter in a range of about 50 μm to about 1000 μm; about 100 μm to about 750 μm; about 125 μm to about 600 μm; about 150 μm to about 500 μm; about 200 μm to about 400 μm; about 200 μm to about 300 μm; or about 150 μm to about 300 μm. On a microscale level, due to the scale of the fiber compared to the cells (e.g., the fiber diameters being larger than the cells), the surface of monofilament fiber is presented as an approximation of a 2D surface for adherent cells to attach and proliferate. Fibers can be woven into a mesh with openings ranging from about 100 μm×100 μm to about 1000 μm×1000 μm. In some embodiments, the opening may have a diameter of about 50 μm to about 1000 μm; about 100 μm to about 750 μm; about 125 μm to about 600 μm; about 150 μm to about 500 μm; about 200 μm to about 400 μm; or about 200 μm to about 300 μm. These ranges of the filament diameters and opening diameters are examples of some embodiments, but are not intended to limit the possible feature sizes of the mesh according to all embodiments. The combination of fiber diameter and opening diameter is chosen to provide efficient and uniform fluid flow through the substrate when, for example, the cell culture matrix comprises a number of adjacent mesh layers (e.g., a stack of individual layers or a rolled mesh layer).

Factors such as the fiber diameter, opening diameter, and weave type/pattern will determine the surface area available for cell attachment and growth. In addition, when the cell culture matrix includes a stack, roll, or other arrangement of overlapping substrate, the packing density of the cell culture matrix will impact the surface area of the packed bed matrix. Packing density can vary with the packing thickness of the substrate material (e.g., the space needed for a layer of the substrate). For example, if a stack of cell culture matrix has a certain height, each layer of the stack can be said to have a packing thickness determined by dividing the total height of the stack by the number of layers in the stack. The packing thickness will vary based on fiber diameter and weave, but can also vary based the alignment of adjacent layers in the stack. For instance, due to the three-dimensional nature of a woven layer, there is a certain amount of interlocking or overlapping that adjacent layers can accommodate based on their alignment with one another. In a first alignment, the adjacent layers can be tightly nestled together, but in a second alignment, the adjacent layers can have zero overlap, such as when the lower-most point of the upper layer is in direct contact with the upper-most point of the lower layer. It may be desirable for certain applications to provide a cell culture matrix with a lower density packing of layers (e.g., when higher permeability is a priority) or a higher density of packing (e.g., when maximizing substrate surface area is a priority). According to one or more embodiments, the packing thickness can be from about 50 μm to about 1000 μm; about 100 μm to about 750 μm; about 125 μm to about 600 μm; about 150 μm to about 500 μm; about 200 μm to about 400 μm; about 200 μm to about 300 μm.

The above structural factors can determine the surface area of a cell culture matrix, whether of a single layer of cell culture substrate or of a cell culture matrix having multiple layers of substrate). For example, in a particular embodiment, a single layer of woven mesh substrate having a circular shape and diameter of 6 cm can have an effective surface area of about 68 cm². The “effective surface area,” as used herein, is the total surface area of fibers in a portion of substrate material that is available for cell attachment and growth. Unless stated otherwise, references to “surface area” refer to this effective surface area. According to one or more embodiments, a single woven mesh substrate layer with a diameter of 6 cm may have an effective surface area of about 50 cm² to about 90 cm²; about 53 cm² to about 81 cm²; about 68 cm²; about 75 cm²; or about 81 cm². These ranges of effective surface area are provided for example only, and some embodiments may have different effective surface areas. The cell culture matrix can also be characterized in terms of porosity, as discussed in the Examples herein.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different patterns or weaves, including, for example knitted, warp-knitted, or woven (e.g., plain weave, twilled weave, dutch weave, five needle weave).

The surface chemistry of the mesh filaments may need to be modified to provide desired cell adhesion properties. Such modifications can be made through the chemical treatment of the polymer material of the mesh or by grafting cell adhesion molecules to the filament surface. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry. In one or more embodiments, however, the mesh is capable of providing an efficient cell growth surface without surface treatment.

The system 50 of FIG. 1 includes a bioreactor 13 housing the cell culture matrix of one or more embodiments disclosed herein. The bioreactor 13 can be fluidly connected to a media conditioning vessel 100, as described above, and the system is capable of supplying a cell culture media within the conditioning vessel 100 to the bioreactor 13. The media conditioning vessel 100 can include sensors and control components found in typical bioreactor used in the bioprocessing industry for a suspension batch, fed-batch or perfusion culture. These include but are not limited to DO oxygen sensors, pH sensors, oxygenator/gas sparging unit, temperature probes, and nutrient addition and base addition ports. A gas mixture supplied to sparging unit can be controlled by a gas flow controller for N₂, O₂, and CO₂ gasses. The media conditioning vessel 100 can also contain a pump or an impeller for media mixing. All media parameters measured by sensors listed above can be controlled by a media conditioning control unit in communication with the media conditioning vessel 100, and capable of measuring and/or adjusting the conditions of the cell culture media to the desired levels. As shown in FIG. 1, the media conditioning vessel 100 is provided as a vessel that is separate from the bioreactor vessel 13. This can have advantages in terms of being able to condition the media separate from where the cells are cultured, and then supplying the conditioned media to the cell culture space.

The media from the media conditioning vessel 100 is delivered to the bioreactor 13 via a connector or tubing 104, which may also include an injection port for cell inoculum to seed and begin culturing of cells. The bioreactor vessel 13 may also include on or more outlets to another connector or tubing 105 through which the cell culture media exits the vessel 13. To analyze the contents of the outflow from the bioreactor 13, one or more sensors may be provided in the line. In some embodiments, the system 50 includes a flow control unit for controlling the flow into and/or out of the bioreactor 13 and/or media conditioning system 100. For example, the flow control unit may receive a signal from the one or more sensors (e.g., an 02 sensor) and, based on the signal, adjust the flow into the bioreactor 13 by sending a signal to a pump (e.g., peristaltic pump) upstream of the inlet to the bioreactor 13. Thus, based on one or a combination of factors measured by the sensors, the pump can control the flow into the bioreactor 13 to obtain the desired cell culturing conditions.

The media perfusion rate is controlled by the signal processing unit that collects and compares sensors signals from media conditioning system 100 and sensors located, for example, within or at the outlet of the bioreactor 13. Because of the pack flow nature of media perfusion through the packed bed bioreactor, nutrients, pH and oxygen gradients are developed along the packed bed. The perfusion flow rate of the bioreactor can be automatically controlled by the flow control unit operably connected to the peristaltic pump.

Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a media conditioning vessel for a perfusion bioreactor system, the media conditioning vessel comprising: a vessel comprising an interior cavity configured to hold a volume of liquid cell culture media; a media inlet configured to return cell culture media from a perfusion bioreactor to the vessel; and a media outlet configured to transfer cell culture media out of the vessel to the perfusion bioreactor, wherein at least one of the media inlet and the media outlet comprises one or more inline sensors configured to measure or detect a characteristic of the cell culture media.

Aspect 2 pertains to the media conditioning vessel of Aspect 1, wherein the one or more inline sensors comprises at least one of a dissolved oxygen sensor, a pH sensor, and a temperature sensor.

Aspect 3 pertains to the media conditioning vessel of Aspect 1 or Aspect 2, further comprising a gas sparging tube configured to sparge a gas in the interior cavity.

Aspect 4 pertains to the media conditioning vessel of any one of Aspects 1-3, further comprising a perfusion loop comprising a pump configured to pump cell culture media from the media outlet to a media return that returns cell culture media to the interior cavity.

Aspect 5 pertains to the media conditioning vessel of Aspect 4, wherein the one or more inline sensors are disposed inline in the perfusion loop.

Aspect 6 pertains to the media conditioning vessel of Aspect 4 or Aspect 5, further comprising a supply tube configured to supply media to the perfusion bioreactor, the supply tube being disposed between the media outlet and the one or more inline sensors in the perfusion loop.

Aspect 7 pertains to the media conditioning vessel of any one of the preceding Aspects, further comprising one or more patch sensors attached to a sidewall or a bottom of the media conditioning vessel.

Aspect 8 pertains to the media conditioning vessel of Aspect 7, wherein the one or more patch sensors comprises at least one of a dissolved oxygen sensor, a pH sensor, and a temperature sensor.

Aspect 9 pertains to the media conditioning vessel of any one of Aspects 1-8, further comprising an outer sparge tube disposed within the interior cavity, wherein the media inlet comprises a tube within the interior cavity, the tube being at least partially disposed within the outer sparge tube, and wherein a gas inlet is at least partially disposed within the outer sparge tube.

Aspect 10 pertains to the media conditioning vessel of Aspect 9, wherein the outer sparge tube comprises a sidewall comprising a plurality of openings sized to prevent bubbles from within the outer sparge tube passing through the plurality of openings.

Aspect 11 pertains to a media conditioning vessel for a perfusion bioreactor system, the media conditioning vessel comprising: a vessel comprising an interior cavity configured to hold a volume of liquid cell culture media; a media inlet configured to return cell culture media from a perfusion bioreactor to the vessel; a media outlet configured to transfer cell culture media out of the vessel to the perfusion bioreactor; and one or more patch sensors attached to at least one of a sidewall or a bottom of the media conditioning vessel and configured to measure or detect a characteristic of the cell culture media.

Aspect 12 pertains to the media conditioning vessel of Aspect 11, wherein the one or more patch sensors comprises at least one of a dissolved oxygen sensor, a pH sensor, and a temperature sensor.

Aspect 13 pertains to the media conditioning vessel of Aspect 11 or Aspect 12, further comprising a gas sparging tube configured to sparge a gas in the interior cavity.

Aspect 14 pertains to the media conditioning vessel of any one of Aspects 11-13, wherein at least one of the media inlet and the media outlet comprises one or more inline sensors configured to measure or detect a characteristic of the cell culture media.

Aspect 15 pertains to the media conditioning vessel of Aspect 14, wherein the one or more inline sensors comprises at least one of a dissolved oxygen sensor, a pH sensor, and a temperature sensor.

Aspect 16 pertains to the media conditioning vessel of any one of Aspects 11-15, further comprising a perfusion loop comprising a pump configured to pump cell culture media from the media outlet to a media return that returns cell culture media to the interior cavity.

Aspect 17 pertains to the media conditioning vessel of Aspect 16, wherein the one or more inline sensors are disposed inline in the perfusion loop.

Aspect 18 pertains to the media conditioning vessel of Aspect 16 or Aspect 17, further comprising a supply tube configured to supply media to the perfusion bioreactor, the supply tube being disposed between the media outlet and the one or more inline sensors in the perfusion loop.

Aspect 19 pertains to the media conditioning vessel of any one of Aspects 11-18, further comprising an outer sparge tube disposed within the interior cavity, wherein the media inlet comprises a tube within the interior cavity, the tube being at least partially disposed within the outer sparge tube, and wherein a gas inlet is at least partially disposed within the outer sparge tube.

Aspect 20 pertains to the media conditioning vessel of Aspect 19, wherein the outer sparge tube comprises a sidewall comprising a plurality of openings sized to prevent bubbles from within the outer sparge tube passing through the plurality of openings.

Aspect 21 pertains to a cell culture system comprising: a bioreactor comprising a first vessel configured to contain a cell culture substrate for adherent-based cells; and a second vessel configured for conditioning cell culture media, the second vessel being in fluid communication with the first vessel via one or more fluid flow connectors, wherein the second vessel is configured for overlay gassing of cell culture media contained therein, and the second vessel comprises a geometry suitable for conditioning different volumes of cell culture media depending on the size of the bioreactor and the cell culture substrate.

Aspect 22 pertains to the cell culture system of Aspect 21, further comprising one or more sensors configured to measure or detect a characteristic of the cell culture media.

Aspect 23 pertains to the cell culture system of Aspect 22, wherein the one or more sensors are inline sensors disposed in a fluid inlet through which cell culture media enters into the second vessel or in a fluid outlet through which cell culture media exits the second vessel.

Aspect 24 pertains to the cell culture system of Aspect 22 or Aspect 23, wherein the one or more sensors comprise patch sensors attached to at least one of a sidewall and a bottom of the second vessel.

Aspect 25 pertains to the cell culture system of any one of Aspects 22-24, further comprising at least one of a gas inlet and a cell nutrient inlet connected to the second vessel and configured to supply at least one of a gas and a cell nutrient to the second vessel.

Aspect 26 pertains to the cell culture system of Aspect 25, further comprising at least one of a gas supply connected to the second vessel via the gas inlet and a cell nutrient supply connected to the second vessel via the cell nutrient inlet.

Aspect 27 pertains to the cell culture system of any one of Aspects 21-26, wherein the first vessel and the second vessel are arranged in a perfusion loop, the perfusion loop comprising a fresh media supply line configured to transport cell culture media from the second vessel to the first vessel, and a waste media supply line configured to transport cell culture media from the first vessel to the second vessel.

Aspect 28 pertains to the cell culture system of any one of Aspects 21-27, wherein the second vessel comprises an interior cavity configured to contain the cell culture media, the interior cavity comprising a first diameter in a first portion of the interior cavity and a second diameter in a second portion of the interior cavity, the second diameter being larger than the first diameter.

Aspect 29 pertains to the cell culture system of Aspect 28, wherein the first portion is lower in the interior cavity than the second portion such that the second portion must be filled with a fluid before the second portion can be filled with a fluid.

Aspect 30 pertains to the cell culture system of Aspect 28 or Aspect 29, wherein the interior cavity comprises an inverted conical portion.

Aspect 31 pertains to the cell culture system of any one of Aspects 28-30, wherein the first portion comprises a constant diameter with vertical sidewalls and the second portion comprises a variable diameter with slanted sidewalls.

Aspect 32 pertains to the cell culture system of Aspect 31, wherein the interior cavity further comprises a third portion with a third diameter, the third diameter being equal to or greater than a widest diameter of the second portion, and the second portion being disposed between the first portion and the third portion.

Aspect 33 pertains to the cell culture system of Aspect 32, wherein the first portion comprises vertical sidewalls and a constant first diameter, the second portion comprises slanted sidewalls with a varying diameter that increases from the first diameter to a second diameter, and the third portion comprises vertical sidewall.

Aspect 34 pertains to the cell culture system of any one of Aspect 28-30, wherein the first portion comprises a constant first diameter with vertical sidewalls and the second portion comprises a constant second diameter with vertical sidewalls.

Aspect 35 pertains to the cell culture system of Aspect 34, wherein the interior cavity further comprises a third portion with a third diameter that is larger than the second diameter.

Aspect 36 pertains to the cell culture system of any one of Aspects 28-35, wherein the interior cavity comprises a working volume of from about 0.3 L to about 35 L.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A media conditioning vessel for a perfusion bioreactor system, the media conditioning vessel comprising:
a vessel comprising an interior cavity configured to hold a volume of liquid cell culture media;
a media inlet configured to return cell culture media from a perfusion bioreactor to the vessel; and
a media outlet configured to transfer cell culture media out of the vessel to the perfusion bioreactor,
wherein at least one of the media inlet and the media outlet comprises one or more inline sensors configured to measure or detect a characteristic of the cell culture media.

2. The media conditioning vessel of claim 1, wherein the one or more inline sensors comprises at least one of a dissolved oxygen sensor, a pH sensor, and a temperature sensor.

3. The media conditioning vessel of claim 1, further comprising a gas sparging tube configured to sparge a gas in the interior cavity.

4. The media conditioning vessel of claim 1, further comprising a perfusion loop comprising a pump configured to pump cell culture media from the media outlet to a media return that returns cell culture media to the interior cavity.

5. The media conditioning vessel of claim 4, wherein the one or more inline sensors are disposed inline in the perfusion loop.

6. The media conditioning vessel of claim 4, further comprising a supply tube configured to supply media to the perfusion bioreactor, the supply tube being disposed between the media outlet and the one or more inline sensors in the perfusion loop.

7. The media conditioning vessel of claim 1, further comprising one or more patch sensors attached to a sidewall or a bottom of the media conditioning vessel.

8. The media conditioning vessel of claim 7, wherein the one or more patch sensors comprises at least one of a dissolved oxygen sensor, a pH sensor, and a temperature sensor.

9. The media conditioning vessel of claim 1, further comprising an outer sparge tube disposed within the interior cavity,
wherein the media inlet comprises a tube within the interior cavity, the tube being at least partially disposed within the outer sparge tube, and
wherein a gas inlet is at least partially disposed within the outer sparge tube.

10. The media conditioning vessel of claim 9, wherein the outer sparge tube comprises a sidewall comprising a plurality of openings sized to prevent bubbles from within the outer sparge tube passing through the plurality of openings.

11. A media conditioning vessel for a perfusion bioreactor system, the media conditioning vessel comprising:
a vessel comprising an interior cavity configured to hold a volume of liquid cell culture media;
a media inlet configured to return cell culture media from a perfusion bioreactor to the vessel;
a media outlet configured to transfer cell culture media out of the vessel to the perfusion bioreactor; and
one or more patch sensors attached to at least one of a sidewall or a bottom of the media conditioning vessel and configured to measure or detect a characteristic of the cell culture media.

12. The media conditioning vessel of claim 11, wherein the one or more patch sensors comprises at least one of a dissolved oxygen sensor, a pH sensor, and a temperature sensor.

13. The media conditioning vessel of claim 11, further comprising a gas sparging tube configured to sparge a gas in the interior cavity.

14. The media conditioning vessel of claim 11, wherein at least one of the media inlet and the media outlet comprises one or more inline sensors configured to measure or detect a characteristic of the cell culture media.

15. The media conditioning vessel of claim 14, wherein the one or more inline sensors comprises at least one of a dissolved oxygen sensor, a pH sensor, and a temperature sensor.

16. The media conditioning vessel of claim 11, further comprising a perfusion loop comprising a pump configured to pump cell culture media from the media outlet to a media return that returns cell culture media to the interior cavity.

17. The media conditioning vessel of claim 16, wherein the one or more inline sensors are disposed inline in the perfusion loop.

18. The media conditioning vessel of claim 16, further comprising a supply tube configured to supply media to the perfusion bioreactor, the supply tube being disposed between the media outlet and the one or more inline sensors in the perfusion loop.

19. The media conditioning vessel of claim 11, further comprising an outer sparge tube disposed within the interior cavity,
wherein the media inlet comprises a tube within the interior cavity, the tube being at least partially disposed within the outer sparge tube, and
wherein a gas inlet is at least partially disposed within the outer sparge tube.

20. The media conditioning vessel of claim 19, wherein the outer sparge tube comprises a sidewall comprising a plurality of openings sized to prevent bubbles from within the outer sparge tube passing through the plurality of openings.
